(19) United States
(12) Patent Application Publication
(10) Pub. No.: US 2010/0205908 A1
(43) Pub. Date: Aug. 19, 2010

(54) CONTAINER FILLING PLANT, SUCH AS A BOTTLE OR CAN FILLING PLANT, HAVING EQUIPMENT FOR TREATING CONTAINERS AND A METHOD OF OPERATING SUCH EQUIPMENT

(76) Inventors: Daryoush SANGI, Hamburg (DE); Thomas HEROLD, Ahrensburg (DE)

Correspondence Address: NILS H. LJUNGMAN & ASSOCIATES, P. O. BOX 130, GREENSBURG, PA, 15601-0130, US

(21) Appl. No.: 12/707,431

(22) Filed: Feb. 17, 2010

Related U.S. Application Data
(63) Continuation-in-part of application No. PCT/EP2008/006116, filed on Jul. 25, 2008.

(30) Foreign Application Priority Data
Aug. 17, 2007 (DE) 10 2007 039 008.6

(51) Int. Cl. B65B 55/04; B65B 55/00

(52) U.S. Cl. 53/426; 53/167

(57) ABSTRACT

A container filling plant, such as a bottle or can filling plant, having equipment for treating containers and a method of operating such equipment. The abstract of the disclosure is submitted herewith as required by 37 C.F.R. §1.72(b). As stated in 37 C.F.R. §1.72(b): A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims. Therefore, any statements made relating to the abstract are not intended to limit the claims in any manner and should not be interpreted as limiting the claims in any manner.

### CONTINUING APPLICATION DATA

This application is a Continuation-In-Part application of International Patent Application No. PCT/EP2008/006116, filed on Jul. 25, 2008, which claims priority from Federal Republic of Germany Patent Application No. 10 2007 039 008.6, filed on Aug. 17, 2007. International Patent Application No. PCT/EP2008/006116 was pending as of the filing date of this application. The United States was an elected state in International Patent Application No. PCT/EP2008/006116.

### BACKGROUND

1. Technical Field

The present application relates to a container filling plant, such as a bottle or can filling plant, having equipment for treating containers and a method of operating such equipment.

2. Background Information

Background information is for informational purposes only and does not necessarily admit that subsequently mentioned information and publications are prior art.

A beverage bottling plant for filling bottles with a liquid beverage filling material can possibly comprise a beverage filling machine, which is often a rotary filling machine, with a plurality of beverage filling positions, each beverage filling position having a beverage filling device for filling bottles with liquid beverage filling material. The filling devices may have an apparatus designed to introduce a predetermined volume of liquid beverage filling material into the interior of bottles to a substantially predetermined level of liquid beverage filling material.

Some beverage bottling plants may possibly comprise filling arrangements that receive a liquid beverage material from a toroidal or annular vessel, in which a supply of liquid beverage material is stored under pressure by a gas. The toroidal vessel may also be connected to at least one external reservoir or supply of liquid beverage material by a conduit or supply line. In some circumstances it may even be possible that a beverage bottling plant has two external supply reservoirs, each of which may be configured to store either the same liquid beverage product or different products. These reservoirs could possibly be connected to the toroidal or annular vessel by corresponding supply lines, conduits, or other arrangements. It is also possible that the external supply reservoirs could be in the form of simple storage tanks, or in the form of liquid beverage product mixers.

A wide variety of types of filling elements are used in filling machines in beverage bottling or container filling plants for dispensing a liquid product into bottles, cans or similar containers, including but not limited to filling processes that are carried out under counterpressure for the bottling of carbonated beverages. The apparatus designed to introduce a predetermined flow of liquid beverage filling material further comprises an apparatus that is designed to terminate the filling of the beverage bottles upon the liquid beverage filling material reaching the predetermined level in bottles. There may also be provided a conveyer arrangement that is designed to move bottles, for example, from an inspecting machine to the filling machine.

After a filling process has been completed, the filled beverage bottles are transported or conveyed to a closing machine, which is often a rotary closing machine. A revolving or rotary machine comprises a rotor, which revolves around a central, vertical machine axis. There may further be provided a conveyer arrangement configured to transfer filled bottles from the filling machine to the closing station. A transporting or conveying arrangement can utilize transport star wheels as well as linear conveyors. A closing machine closes bottles by applying a closure, such as a screw-top cap or a bottle cork, to a corresponding bottle mouth. Closed bottles are then usually conveyed to an information adding arrangement, wherein information, such as a product name or a manufacturer's information or logo, is applied to a bottle. A closing station and information adding arrangement may be connected by a corresponding conveyer arrangement. Bottles are then sorted and packaged for shipment out of the plant.

Many beverage bottling plants may also possibly comprise a rinsing arrangement or rinsing station to which new, non-return and/or even return bottles are fed, prior to being filled, by a conveyer arrangement, which can be a linear conveyor or a combination of a linear conveyor and a starwheel. Downstream of the rinsing arrangement or rinsing station, in the direction of travel, rinsed bottles are then transported to the beverage filling machine by a second conveyer arrangement that is formed, for example, by one or more starwheels that introduce bottles into the beverage filling machine.

In some beverage bottling plants, a treatment machine, treating arrangement, or treatment station may be used to treat beverage bottles or other such containers prior to filling. In these plants, the beverage bottles or other such containers may be treated with a treatment medium, for example a treatment medium like hydrogen peroxide.

It is a further possibility that a beverage bottling plant for filling bottles with a liquid beverage filling material can be controlled by a central control arrangement, which could be, for example, a computerized control system that monitors and controls the operation of the various stations and mechanisms of the beverage bottling plant.

The present application relates to a metering and supplying system for the metered delivery of hydrogen peroxide to treatment heads of a device for the H₂O₂ sterilization of packaging means. The metering and supplying system comprises at least one reservoir, which provides the hydrogen peroxide, and a connecting line having a controllable metering element and extending between the respective treatment head and the reservoir. The present application also relates to a device for the H₂O₂ sterilization of packaging means. The device comprises at least one treatment head for delivering a heated H₂O₂ sterilization medium comprised of hydrogen peroxide and a vaporous and/or gaseous carrier into the packaging means to be sterilized and for subsequently activating the H₂O₂ sterilization medium. The device comprises a metering and supplying system for the metered delivery of the hydrogen peroxide to the at least one treatment head. The present application also relates to a method for operating a device for the H₂O₂ sterilization of packaging means, comprising at least one treatment head for delivering an H₂O₂ sterilization medium comprised of hydrogen peroxide and a vaporous and/or gaseous carrier to the packaging means to be sterilized and for subsequently activating the H₂O₂ sterilization medium. The device, being operated by the method, comprises a metering and supplying system for the metered delivery of the hydrogen peroxide to the treatment heads via controllable connections.

“H₂O₂ sterilization” within the context of the present application refers to the sterilization of packaging means using a sterilization medium that comprises hydrogen peroxide (H₂O₂), or using a sterilization medium comprised solely of H₂O₂ (hereinafter also called H₂O₂ sterilization medium).

“Packaging means” within the context of the present application are for example bottles or similar containers, including KEGs, cans, tubes, and other types of packages, including soft packages or bags, which must be or should be sterilized before being filled with product, for example foods or products for consumption, such as beverages or pharmaceuticals.

Methods and devices for sterilizing packaging means using an H₂O₂ sterilization medium, which comprises hydrogen peroxide in a mixture with hot sterile air and which is formed in treatment heads of the respective H₂O₂ sterilization device, are known in the art. In these methods, an H₂O₂ condensation film is formed via condensation of the hot H₂O₂ sterilization medium, which is introduced into the packaging means through the respective treatment head, on the interior surface of the cooler packaging means, after which said condensation film is activated in a subsequent activation phase by introducing a sterile, hot gaseous and/or vaporous activation medium, for example by introducing hot sterile air, so that in the decomposition of H₂O₂, oxygen free radicals are created, which react with any bacteria and contaminants that are present, thereby sterilizing the packaging means.

To produce high-quality H₂O₂ sterilization, i.e., for example with a high sterilization rate that can be reproduced, and thereby to minimize the amount of hydrogen peroxide that is consumed, the amount of hydrogen peroxide added to the treatment heads that are used during formation of the H₂O₂ sterilization medium must be or should be precisely or substantially precisely or generally metered.

The present application relates to a metering and supplying system for devices for the H₂O₂ sterilization of packaging means, device with a metering and supplying system of this type, and method for operating such a device.

### OBJECT OR OBJECTS

An object of the present application is to specify a metering and supplying system which will enable optimum hydrogen peroxide metering with reproducible results. A further object of the present application is to specify a method for precisely or substantially precisely or generally verifying completion of the delivery of the predetermined quantity of H₂O₂.

### SUMMARY

To attain this object, the present application discloses a metering and supplying system for the metered delivery of hydrogen peroxide to treatment heads of a device for the H₂O₂ sterilization of packaging means. The metering and supplying system comprises at least one reservoir, which provides the hydrogen peroxide, and a connecting line having a controllable metering element and extending between the respective treatment head and the reservoir. Each treatment head is provided with its own connecting line which leads to a reservoir or to a distributing line. The metering element is a metering valve, which can be electrically controlled at least during metering between an opened state and a closed state, and is provided at the treatment head or directly or substantially directly on the treatment head. The present application also discloses a device for the H₂O₂ sterilization of packaging means. The device comprises at least one treatment head for delivering a heated H₂O₂ sterilization medium comprised of hydrogen peroxide and a vaporous and/or gaseous carrier into the packaging means to be sterilized and for subsequently activating the H₂O₂ sterilization medium. The device also comprises a metering and supplying system for the metered a delivery of the hydrogen peroxide to the at least one treatment head. The metering and supplying system is embodied according to the present application. The present application also discloses a method for operating a device for the H₂O₂ sterilization of packaging means, comprising at least one treatment head for delivering an H₂O₂ sterilization medium comprised of hydrogen peroxide and a vaporous and/or gaseous carrier to the packaging means to be sterilized and for subsequently activating the H₂O₂ sterilization medium. The device, being operated in the method, and comprising a metering and supplying system for the metered delivery of the hydrogen peroxide to the treatment heads via controllable connections. During operation, metering valves provided as metering elements in the controlled connections are continually opened and closed in such a way that a maximum residence time for the hydrogen peroxide in the controllable connections of sixty seconds, in one possible embodiment a maximum residence time of forty seconds, results.

Further improvements, embodiments and potential applications for the present application are specified in the following description of possible embodiments and in the set of drawings. In these components, described and/or illustrated characterizing features, alone or in any combination, constitute the subject of the present application.

The above-discussed embodiments of the present invention will be described further herein below. When the word “invention” or “embodiment of the invention” is used in this specification, the word “invention” or “embodiment of the invention” includes “inventions” or “embodiments of the invention”, that is the plural of “invention” or “embodiment of the invention”. By stating “invention” or “embodiment of the invention”, the Applicant does not in any way admit that the present application does not include more than one patentably and non-obviously distinct invention, and maintains that this application may include more than one patentably and non-obviously distinct invention. The Applicant hereby asserts that the disclosure of this application may include more than one invention, and, in the event that there is more than one invention, that these inventions may be patentable and non-obvious one with respect to the other.

### BRIEF DESCRIPTION OF THE DRAWINGS

In what follows, the present application will be disclosed in greater detail in reference to the set of drawings of possible embodiments:

FIG. 1 shows a highly simplified illustration of a plan view of a machine or device for H₂O₂ sterilization of packaging means in the form of bottles;

FIG. 2 shows a simplified illustration of a treatment head of the device of FIG. 1;

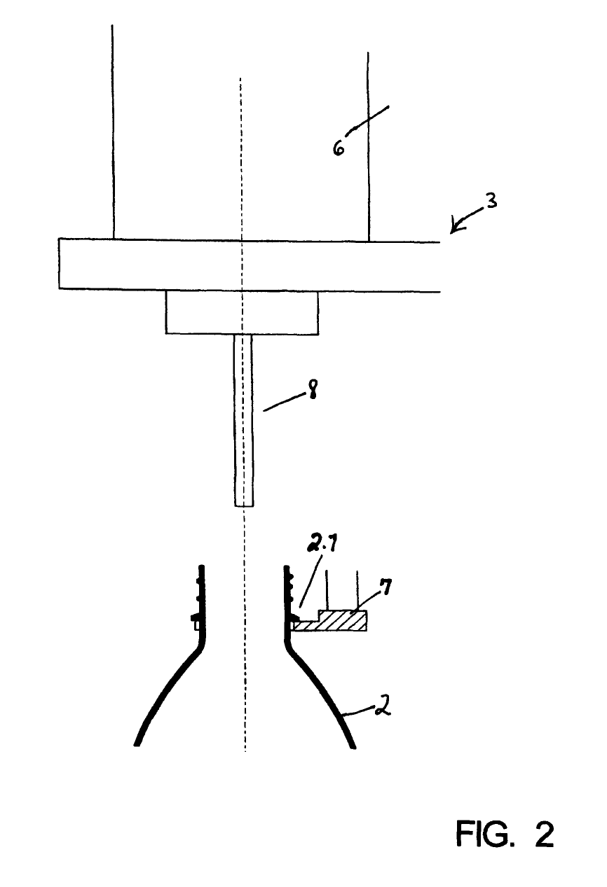

FIG. 3 shows a functional representation of a system for the metered supplying of hydrogen peroxide to multiple treatment heads of the device of FIG. 1;

FIG. 3A shows another functional representation of a system for the metered supplying of hydrogen peroxide to multiple treatment heads of the device of FIG. 1;

FIG. 4 shows another functional representation of a system for the metered supplying of hydrogen peroxide to multiple treatment heads of the device of FIG. 1;

FIG. 5 shows another functional representation of a system for the metered supplying of hydrogen peroxide to multiple treatment heads of the device of FIG. 1; and

FIG. 6 shows schematically the main components of one possible embodiment example of a system for filling containers, for example a beverage bottling plant for filling bottles with at least one liquid beverage, in accordance with at least one possible embodiment, in which system or plant could possibly be utilized at least one aspect, or several aspects, of the embodiments disclosed herein.

### DESCRIPTION OF EMBODIMENT OR EMBODIMENTS

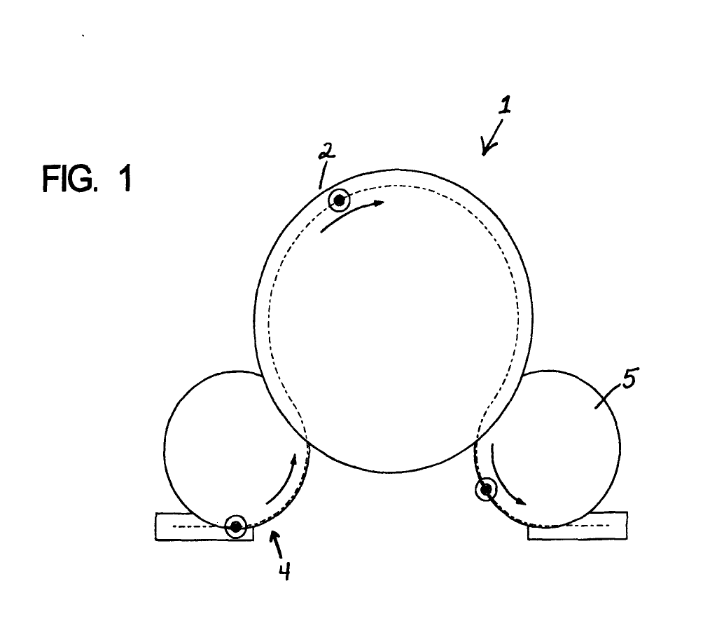

FIG. 1 shows a device for the treatment or sterilization of bottles using hydrogen peroxide (H₂O₂). The device is comprised essentially of a rotor 3, which is driven to rotate about a vertical axis, with the bottles 2 to be treated and/or sterilized being fed to said rotor via a container intake 4, and with the treated and/or sterilized bottles 2 being removed from said rotor via a container outlet 5 and then fed to a further application, for example to a filler for filling with a product.

A plurality of treatment heads 6 are provided around the periphery of the rotor 3, spaced at equal angular distances about the vertical machine or rotor axis. Each treatment head on the rotor is assigned a bottle or container carrier 7, which holds the respective bottle 2 underneath the treatment head 6 during treatment, with the bottles 2 in the embodiment illustrated here, embodied as PET bottles, being shown suspended from an opening flange on the bottle.

The bottles 2 are treated, i.e., sterilized, using the H₂O₂ sterilization medium, which is generated in a known manner inside a mixing chamber 6.1 formed inside the treatment head 6 by spraying hydrogen peroxide, for example thirty-five percent hydrogen peroxide, via at least one mixing nozzle, into sterile air to form a hydrogen peroxide/air aerosol, and then heating this aerosol in an evaporator 6.2 to a temperature of one hundred forty-five degrees Celsius, for example. A port 6.1.1 for the sterile air and another port 6.1.2 for the hydrogen peroxide are provided on the mixing chamber.

In the treatment process, a tube 8, which is part of the treatment head 6, is inserted into the bottle 2 with its axis aligned with the axis of the bottle 2, and in an application phase, the hot H₂O₂ sterilization medium is first introduced into the interior of the bottle 2 via this tube in such a way that an H₂O₂ condensation film forms via condensation on the interior surface of the bottle 2. Following this application phase, in the activation phase, the H₂O₂ condensation film is activated by an application of energy, in one possible embodiment by introducing a hot, sterile gaseous and/or vaporous medium, for example, such as by introducing hot sterile air via the tube 8, so that the resulting decomposition reaction or cleavage of H₂O₂ generates free oxygen radicals, which react with bacteria and/or contaminants that are present in the respective bottle 2, resulting in sterilization.

To optimize the consumption of hydrogen peroxide while achieving a high-quality sterilization, i.e., sterilization with a high sterilization rate which can be reproduced and verified, a precise or substantially precise or general and in one possible embodiment a reproducible metering of the quantity of hydrogen peroxide that is fed into the respective mixing chamber 6.1 via the port 6.1.2 is essential.

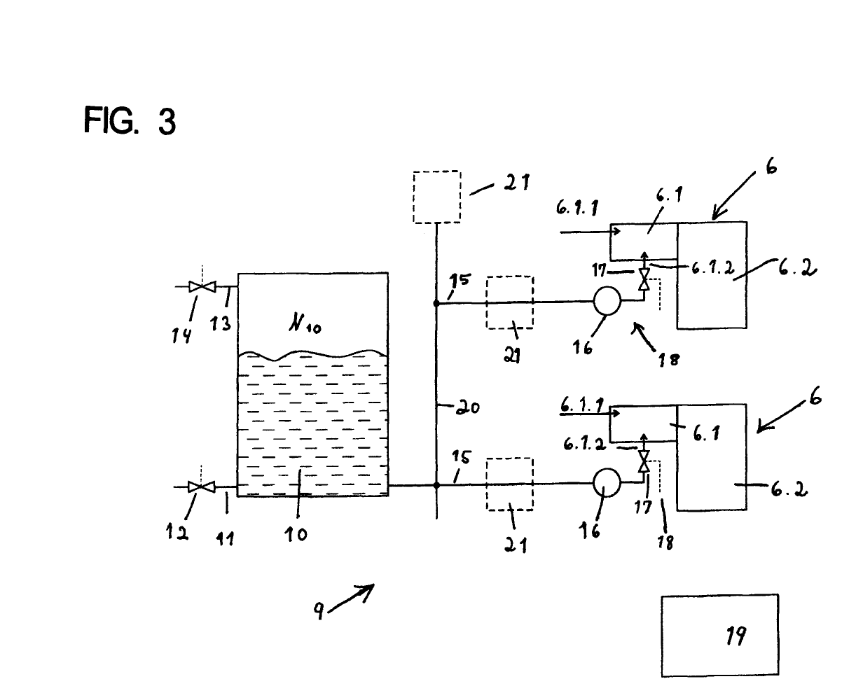

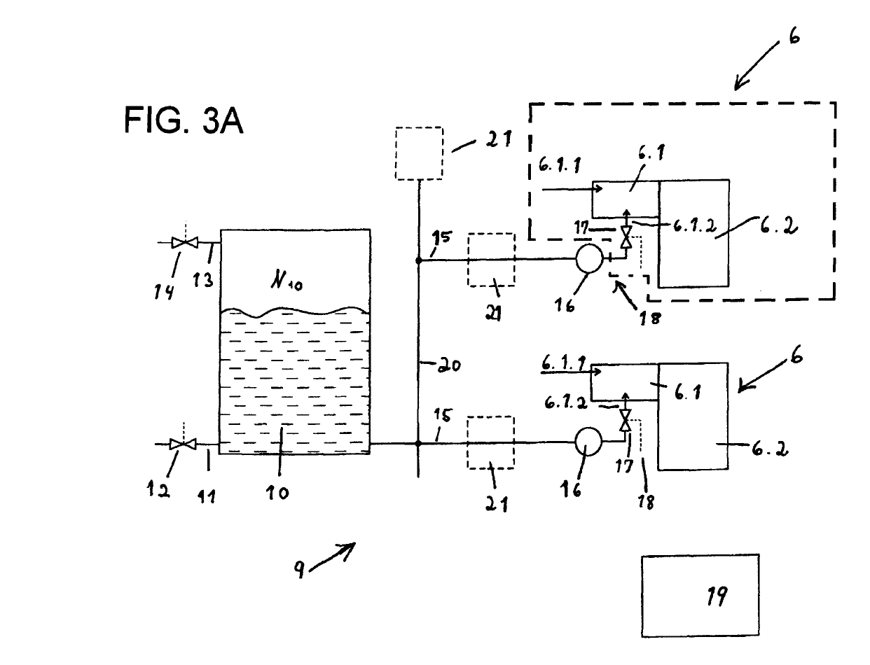

FIG. 3 shows a simplified representation in block diagram form of a supplying and metering system for the supplying and/or metered delivery of hydrogen peroxide to the individual treatment heads 6 of the device of FIG. 1, wherein in FIG. 3 two treatment heads 6 are shown for purposes of simplification.

One principal problem encountered in the metered addition of hydrogen peroxide to the treatment heads 6 is that the H₂O₂, as a less stable product, continuously decomposes into water and oxygen even at normal room temperature, and that this decomposition rate even increases in an uncontrolled fashion as a result of pressure changes and/or pressure surges that can occur in an H₂O₂ conducting system.

According to the knowledge upon which the present application is based, a metered delivery of hydrogen peroxide from a reservoir via metering pumps, such as membrane pumps, cannot be achieved with high metering precision, in part because oxygen bubbles form in pumps of this type and in the lines leading up to and away from the pumps as a result of the decomposition of the hydrogen peroxide, which makes precise or substantially precise or general metering impossible.

This problem is solved by the embodiment of the metering and supplying system, identified as a whole in FIG. 3 by the number 9. The metering and supplying system 9 comprises, in part, a reservoir or supply tank 10, which is filled with the hydrogen peroxide up to a preset level N₁₀ with level control, so that a pressure chamber is formed in the reservoir 10 above the level N₁₀ and a fluid chamber that holds the hydrogen peroxide is formed below the level N₁₀.

The reservoir 10 is connected via a supply line 11 to a control valve 12 on a source which provides pressurized hydrogen peroxide. To generate the metering pressure, the pressure chamber of the reservoir 10 is connected via a supply line 13 to a pressure control valve 14, for example to a reverse venting pressure-reducing valve, on a source which provides pressurized sterile air.

Multiple connecting lines 15 open into the fluid chamber of the reservoir 10, with each line being connected via a flow meter 16, for example via a magnetic induction flow meter and a high-speed metering valve 17, to a treatment head 6 or to the port 6.1.2 of the respective mixing chamber 6.1. The flow meter 16 and the metering valves 17 arranged in series with it together form a metering and dosing unit 18, with one such unit being provided for each treatment head 6 separately, and with the metering valves 17 of said unit being individually controllable via a central control unit 19 based upon the electrical measurement signal supplied by the associated flow meter 16, for example.

Each of the metering valves 17 can be controlled, in one possible embodiment pulse controlled, between an opened state and a closed state via the control unit 19, with the metering being controlled, for example, by modulating the pulse width of the electrical signals that control the metering valves 17 or by adjusting the times between the opened state and the closed state.

To prevent, restrict, and/or minimize the elimination or substantial elimination or reduction of oxygen from the hydrogen peroxide, and the resulting metering errors, in the metering and supplying system 9 the distance traveled by the hydrogen peroxide inside the metering and dosing unit 18, and thus in one possible embodiment the distance between the respective metering valve 17 and the mixing chamber 6.1 or the at least one mixing nozzle assigned there to the port 6.1.2, is very short, thereby also ensuring a very short residence time of the hydrogen peroxide in the metering and dosing unit 18, which prevents, restrict, and/or minimize the elimination or substantial elimination or reduction of oxygen in harmful amounts. For the same reason, the connecting lines 15 are configured as short as possible and with the smallest possible interior cross section.

The connecting lines 15 are in one possible embodiment approximately one and one half meters to two meters in length. The interior cross section of these connecting lines then measures approximately one and one half millimeters, for example. This decreased cross section is in one possible embodiment maintained over the entire controllable connection between the respective treatment head 6 and the reservoir 10, i.e., not only in the connecting lines 15, but also at the metering and dosing units 18, and at the respective elements that form these units. As a result of these or other measures, combined with the periodic actuation of the metering valves 17, during normal operation of the device 1 the residence time of the hydrogen peroxide in the connection between the reservoir 10 and the port 6.1.2 or in the at least one mixing nozzle of the respective treatment head 6 there is kept at a maximum of sixty seconds, in one possible embodiment a maximum of forty seconds. It is also possible that metering pressure is generated not by pumps but by pressurization of the reservoir 10, and in one possible embodiment that high-speed or fast action valves, rather than metering pumps, are used as the metering elements.

In tests conducted in house by the applicant, it was determined that adequate metering precision of the H₂O₂ can be achieved when the metering valves 17 have in one possible embodiment short switching times. It was determined that with a switching time of more than ninety milliseconds, adequate precision cannot be achieved. This is due to the complex flow processes that occur within the overall system during opening and/or closing of the valves. In one possible embodiment, the acceleration or deceleration of the fluid columns located in the lines causes unacceptable metering imprecision with long switching times as a result of the permanently or substantially permanently but inconsistently or substantially inconsistently changing flow rate of the H₂O₂ sterilization medium over an extended period of time. A definite improvement in precision of metering results was achieved with switching times of less than fifty milliseconds.

The optimum metering precision results for the metered quantity of H₂O₂ sterilization medium were achieved with switching times of less than one millisecond. Valves with such short switching times that are suitable for controlling a flow of liquid are known to one of ordinary skill in the art from the field of inkjet printing technology, for example.

The operation of the metering and supplying system 9 is described in the above specification, i.e., for the metered delivery of the hydrogen peroxide at the respective treatment head the relevant metering valve 17 is controlled based upon the metering pressure existing at the metering valve, the control signal supplied by the flow meter 16, and the flow rate of the sterile air flowing into the respective mixing chamber 6.1.

To remove any oxygen that may have been eliminated by the hydrogen peroxide in the connecting lines 15, gas separators 21 can be provided in these connecting lines or in a distributing line 20, via which the connecting lines 15 are connected to the reservoir 10, as is indicated in FIG. 3 by dashed lines.

Above, the present application has been specified within the context of a possible embodiment. Of course, changes and modifications to this may be made, without thereby departing from the teaching concept upon which the present application is based.

For example, it is proposed to regulate the quantity of hydrogen peroxide that is supplied to an individual treatment head by means of a control circuit. In this case it is possible for the parameter combination of metering pressure/metering time to also be taken into consideration as a part of this regulation.

Above, the present application has been specified within the context of the H₂O₂ sterilization of bottles 2. However, the present application, in one possible embodiment the described metering systems 9 and 9a, is also suitable for the H₂O₂ sterilization of other packaging means, such as screw caps, for example.

FIG. 3A may show one possible embodiment of the present application. As seen in FIG. 3A, the treatment head 6 comprises the evaporator 6.2, mixing chamber 6.1, port for sterile air 6.1.1, port for hydrogen peroxide 6.1.2, and metering valve 17. One treatment head 6 is shown with these components, illustrated by the dotted line. In other possible embodiments of the present application, all or substantially all of the treatment heads 6 may comprise the evaporator 6.2, mixing chamber 6.1, port for sterile air 6.1.1, port for hydrogen peroxide 6.1.2, and metering valve 17. In another possible embodiment of the present application, a portion of the treatment heads 6, which are disposed about the periphery of the rotor 3 of the device 1, each comprise the evaporator 6.2, mixing chamber 6.1, port for sterile air 6.1.1, port for hydrogen peroxide 6.1.2, and metering valve 17.

In at least one possible embodiment of the present application, the treatment head 6 and/or the evaporator 6.2 may be disposed immediately above the bottle 2 to be treated. In at least one possible embodiment of the present application, the bottle 2 may be lifted, for example by a cam system, toward the treatment head 6. In another possible embodiment of the present application, the tube 8 may be lowered into the bottle 2. In at least one possible embodiment of the present application, the metering and supplying system 9 of the present application does not comprise pumps. In this embodiment, the system is under pressure. The pressurized reservoir 10 permits the flow of hydrogen peroxide through the connecting lines 15 and to the treatment heads 6.

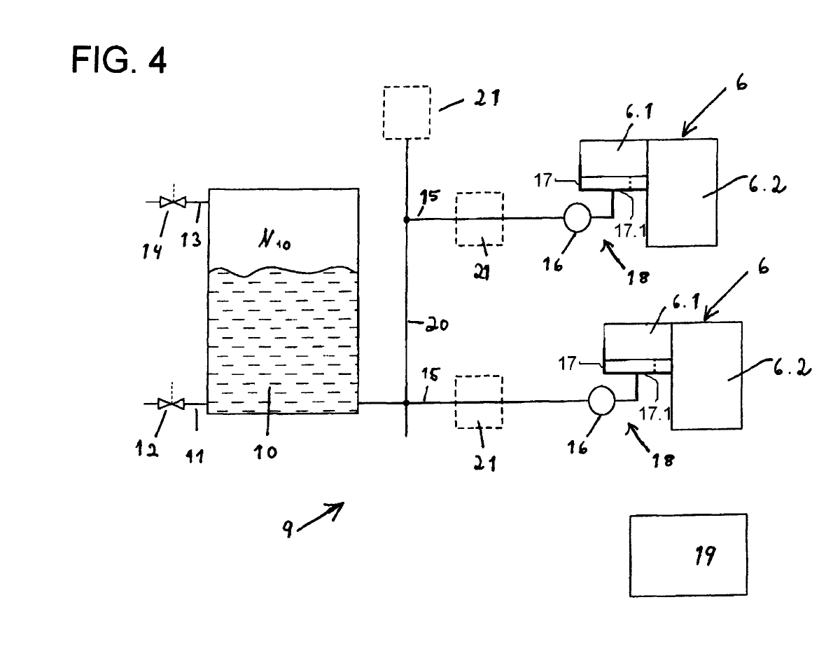

FIG. 4 shows one possible embodiment of a metering and supplying system 9 for use with the treatment machine 1 of the present application. In this possible embodiment, the valve 17 may be connected to, may be adjacent to, or may be directly mounted on the mixing chamber 6.1 and the evaporator 6.2. In another possible embodiment of the present application, the valve 17.1 may be connected to, may be adjacent to, or may be directly mounted on the mixing chamber 6.1, but is not connected to, not adjacent to, or not directly mounted on the evaporator 6.2.

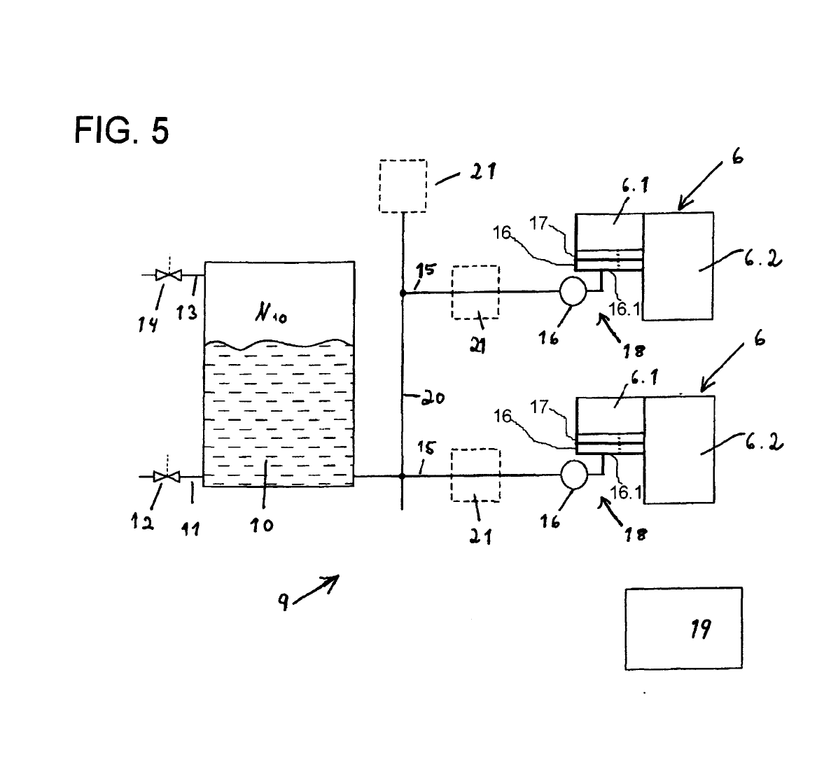

FIG. 5 shows one possible embodiment of a metering and supplying system 9 for use with the treatment machine 1 of the present application. In this possible embodiment, the valve 17 may be connected to, may be adjacent to, or may be directly mounted on the treatment head 6, which also comprises the mixing chamber 6.1 and the evaporator 6.2. In another possible embodiment of the present application, the valve 17.1 may be connected to, may be adjacent to, or may be directly mounted on the mixing chamber 6.1, but is not connected to, not adjacent to, or not directly mounted on the evaporator 6.2.

As also seen in FIG. 5, the flow meter 16 may be connected to, may be adjacent to, or may be directly mounted on the treatment head 6, which comprises the mixing chamber 6.1, evaporator 6.2, and the valve 17 or alternative valve 17.1. In another possible embodiment of the present application, the flow meter 16.1 may be connected to, may be adjacent to, or may be directly mounted on the valve 17.1 but is not connected to, not adjacent to, or not directly mounted on the evaporator 6.2.

In one possible embodiment of the present application, the volume of the connecting lines 15, flow meters 16, and valves 17 may be minimized in order to provide a minimal residence time of hydrogen peroxide in the connecting lines 15, flow meters 16, and valves 17. This minimal residence time may range between about sixty seconds to about forty seconds.

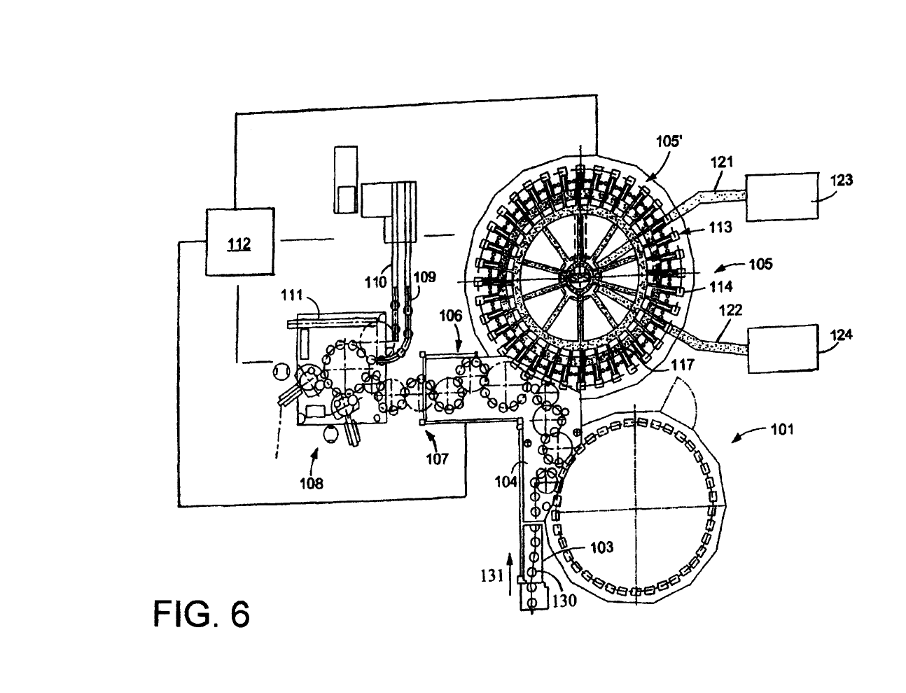

FIG. 6 shows schematically the main components of one possible embodiment example of a system for filling containers, specifically, a beverage bottling plant for filling bottles 130 with at least one liquid beverage, in accordance with at least one possible embodiment, in which system or plant could possibly be utilized at least one aspect, or several aspects, of the embodiments disclosed herein.

FIG. 6 shows a rinsing arrangement or rinsing station 101, to which the containers, namely bottles 130, are fed in the direction of travel as indicated by the arrow 131, by a first conveyer arrangement 103, which can be a linear conveyor or a combination of a linear conveyor and a starwheel. Downstream of the rinsing arrangement or rinsing station 101, in the direction of travel as indicated by the arrow 131, the rinsed bottles 130 are transported to a beverage filling machine 105 by a second conveyer arrangement 104 that is formed, for example, by one or more starwheels that introduce bottles 130 into the beverage filling machine 105.

The beverage filling machine 105 shown is of a revolving or rotary design, with a rotor 105′, which revolves around a central, vertical machine axis. The rotor 105′ is designed to receive and hold the bottles 130 for filling at a plurality of filling positions 113 located about the periphery of the rotor 105′. At each of the filling positions 103 is located a filling arrangement 114 having at least one filling device, element, apparatus, or valve. The filling arrangements 114 are designed to introduce a predetermined volume or amount of liquid beverage into the interior of the bottles 130 to a predetermined or desired level.

The filling arrangements 114 receive the liquid beverage material from a toroidal or annular vessel 117, in which a supply of liquid beverage material is stored under pressure by a gas. The toroidal vessel 117 is a component, for example, of the revolving rotor 105′. The toroidal vessel 117 can be connected by means of a rotary coupling or a coupling that permits rotation. The toroidal vessel 117 is also connected to at least one external reservoir or supply of liquid beverage material by a conduit or supply line. In the embodiment shown in FIG. 6, there are two external supply reservoirs 123 and 124, each of which is configured to store either the same liquid beverage product or different products. These reservoirs 123, 124 are connected to the toroidal or annular vessel 117 by corresponding supply lines, conduits, or arrangements 121 and 122. The external supply reservoirs 123, 124 could be in the form of simple storage tanks, or in the form of liquid beverage product mixers, in at least one possible embodiment.

As well as the more typical filling machines having one toroidal vessel, it is possible that in at least one possible embodiment there could be a second toroidal or annular vessel which contains a second product. In this case, each filling arrangement 114 could be connected by separate connections to each of the two toroidal vessels and have two individually-controllable fluid or control valves, so that in each bottle 130, the first product or the second product can be filled by means of an appropriate control of the filling product or fluid valves.

Downstream of the beverage filling machine 105, in the direction of travel of the bottles 130, there can be a beverage bottle closing arrangement or closing station 106 which closes or caps the bottles 130. The beverage bottle closing arrangement or closing station 106 can be connected by a third conveyer arrangement 107 to a beverage bottle labeling arrangement or labeling station 108. The third conveyor arrangement may be formed, for example, by a plurality of starwheels, or may also include a linear conveyor device.

In the illustrated embodiment, the beverage bottle labeling arrangement or labeling station 108 has at least one labeling unit, device, or module, for applying labels to bottles 130. In the embodiment shown, the labeling arrangement 108 is connected by a starwheel conveyer structure to three output conveyer arrangements: a first output conveyer arrangement 109, a second output conveyer arrangement 110, and a third output conveyer arrangement 111, all of which convey filled, closed, and labeled bottles 130 to different locations.

The first output conveyer arrangement 109, in the embodiment shown, is designed to convey bottles 130 that are filled with a first type of liquid beverage supplied by, for example, the supply reservoir 123. The second output conveyer arrangement 110, in the embodiment shown, is designed to convey bottles 130 that are filled with a second type of liquid beverage supplied by, for example, the supply reservoir 124. The third output conveyer arrangement 111, in the embodiment shown, is designed to convey incorrectly labeled bottles 130. To further explain, the labeling arrangement 108 can comprise at least one beverage bottle inspection or monitoring device that inspects or monitors the location of labels on the bottles 130 to determine if the labels have been correctly placed or aligned on the bottles 130. The third output conveyer arrangement 111 removes any bottles 130 which have been incorrectly labeled as determined by the inspecting device.

The beverage bottling plant can be controlled by a central control arrangement 112, which could be, for example, computerized control system that monitors and controls the operation of the various stations and mechanisms of the beverage bottling plant.

Dosing and supplying system for the dosed feeding of hydrogen peroxide to treatment heads 6 of a device for the H₂O₂-sterilizing of packaging means 2, comprising at least one reservoir 10, providing the hydrogen peroxide, and a connecting line 15, having a controllable dosing element 17 and extending between the respective treatment head 6 and the reservoir 10, wherein each treatment head 6 is provided with its own connecting line 15, leading to a reservoir 10 or to a distributor 20, and wherein the dosing element is a dosing valve 17, which can be electrically controlled, at least during the dosing, between an open state and a closed state and is provided at the treatment head 6 or directly or substantially directly or generally on the treatment head.

One feature or aspect of an embodiment is believed at the time of the filing of this patent application to possibly reside broadly in a metering and supplying system for the metered delivery of hydrogen peroxide to treatment heads 6 of a device for the H₂O₂ sterilization of packaging means 2, comprising at least one reservoir 10 which provides the hydrogen peroxide and a connecting line 15 having a controllable metering element 17 and extending between the respective treatment head 6 and the reservoir 10, wherein each treatment head 6 is provided with its own connecting line 15 which leads to a reservoir 10 or to a distributing line 20, and in that the metering element is a metering valve 17, which can be electrically controlled at least during metering between an opened state and a closed state, and is provided at the treatment head 6 or directly on the treatment head.

Another feature or aspect of an embodiment is believed at the time of the filing of this patent application to possibly reside broadly in the metering and supplying system, wherein a flow meter 16, for example a magnetic induction flow meter (MIF), is provided in series with the respective metering valve 17.

Yet another feature or aspect of an embodiment is believed at the time of the filing of this patent application to possibly reside broadly in the metering and supplying system, wherein the reservoir 10 is a container that is or can be pressurized with overpressure or metering pressure.

Still another feature or aspect of an embodiment is believed at the time of the filing of this patent application to possibly reside broadly in the metering and supplying system, wherein the connecting lines 15 have a maximum length of approximately one and one half meters to two meters.

A further feature or aspect of an embodiment is believed at the time of the filing of this patent application to possibly reside broadly in the metering and supplying system, wherein the connection between the at least one reservoir 10 and the respective treatment head 6 has a cross section of approximately one and a half millimeters over at least most of its length.

Another feature or aspect of an embodiment is believed at the time of the filing of this patent application to possibly reside broadly in the metering and supplying system, wherein the controllable connection between the respective treatment head and the reservoir 10 is embodied such that the residence time of the hydrogen peroxide in the respective controllable connection is a maximum of sixty seconds, in one possible embodiment a maximum of forty seconds.

Yet another feature or aspect of an embodiment is believed at the time of the filing of this patent application to possibly reside broadly in the metering and supplying system, wherein the reservoir 10 can be pressurized with overpressure or metering pressure.

Still another feature or aspect of an embodiment is believed at the time of the filing of this patent application to possibly reside broadly in the metering and supplying system, wherein the metering valves 17 have a switching time of less than ninety milliseconds.

A further feature or aspect of an embodiment is believed at the time of the filing of this patent application to possibly reside broadly in the metering and supplying system, wherein the metering valves 17 have a switching time of less than fifty milliseconds.

Another feature or aspect of an embodiment is believed at the time of the filing of this patent application to possibly reside broadly in the metering and supplying system, wherein the metering valves 17 have a switching time of less than one millisecond.

Yet another feature or aspect of an embodiment is believed at the time of the filing of this patent application to possibly reside broadly in a device for the H₂O₂ sterilization of packaging means 2, comprising at least one treatment head 6 for delivering a heated H₂O₂ sterilization medium comprised of hydrogen peroxide and a vaporous and/or gaseous carrier into the packaging means 2 to be sterilized and for subsequently activating the H₂O₂ sterilization medium, and comprising a metering and supplying system 9 for the metered delivery of the hydrogen peroxide to the at least one treatment head 6, wherein the metering and supplying system 9 is embodied according to the present application.

Still another feature or aspect of an embodiment is believed at the time of the filing of this patent application to possibly reside broadly in a method for operating a device for the H₂O₂ sterilization of packaging means 2, comprising at least one treatment head 6 for delivering an H₂O₂ sterilization medium comprised of hydrogen peroxide and a vaporous and/or gaseous carrier to the packaging means 2 to be sterilized and for subsequently activating the H₂O₂ sterilization medium, and comprising a metering and supplying system 9 for the metered delivery of the hydrogen peroxide to the treatment heads 6 via controllable connections 15, 16, 17, wherein during operation, metering valves 17 provided as metering elements in the controlled connections are continually or substantially continually opened and closed in such a way that a maximum residence time for the hydrogen peroxide in the controllable connections 15, 16, 17 of sixty seconds, in one possible embodiment of forty seconds, results.

The components disclosed in the various publications, disclosed or incorporated by reference herein, may possibly be used in possible embodiments of the present invention, as well as equivalents thereof.

The purpose of the statements about the technical field is generally to enable the Patent and Trademark Office and the public to determine quickly, from a cursory inspection, the nature of this patent application. The description of the technical field is believed, at the time of the filing of this patent application, to adequately describe the technical field of this patent application. However, the description of the technical field may not be completely applicable to the claims as originally filed in this patent application, as amended during prosecution of this patent application, and as ultimately allowed in any patent issuing from this patent application. Therefore, any statements made relating to the technical field are not intended to limit the claims in any manner and should not be interpreted as limiting the claims in any manner.

The appended drawings in their entirety, including all dimensions, proportions and/or shapes in at least one embodiment of the invention, are accurate and are hereby included by reference into this specification.

The background information is believed, at the time of the filing of this patent application, to adequately provide background information for this patent application. However, the background information may not be completely applicable to the claims as originally filed in this patent application, as amended during prosecution of this patent application, and as ultimately allowed in any patent issuing from this patent application. Therefore, any statements made relating to the background information are not intended to limit the claims in any manner and should not be interpreted as limiting the claims in any manner.

All, or substantially all, of the components and methods of the various embodiments may be used with at least one embodiment or all of the embodiments, if more than one embodiment is described herein.

The purpose of the statements about the object or objects is generally to enable the Patent and Trademark Office and the public to determine quickly, from a cursory inspection, the nature of this patent application. The description of the object or objects is believed, at the time of the filing of this patent application, to adequately describe the object or objects of this patent application. However, the description of the object or objects may not be completely applicable to the claims as originally filed in this patent application, as amended during prosecution of this patent application, and as ultimately allowed in any patent issuing from this patent application. Therefore, any statements made relating to the object or objects are not intended to limit the claims in any manner and should not be interpreted as limiting the claims in any manner.

All of the patents, patent applications and publications recited herein, and in the Declaration attached hereto, are hereby incorporated by reference as if set forth in their entirety herein.

The summary is believed, at the time of the filing of this patent application, to adequately summarize this patent application. However, portions or all of the information contained in the summary may not be completely applicable to the claims as originally filed in this patent application, as amended during prosecution of this patent application, and as ultimately allowed in any patent issuing from this patent application. Therefore, any statements made relating to the summary are not intended to limit the claims in any manner and should not be interpreted as limiting the claims in any manner.

It will be understood that the examples of patents, published patent applications, and other documents which are included in this application and which are referred to in paragraphs which state “Some examples of . . . which may possibly be used in at least one possible embodiment of the present application . . . ” may possibly not be used or useable in any one or more embodiments of the application.

The sentence immediately above relates to patents, published patent applications and other documents either incorporated by reference or not incorporated by reference.

Some examples of timer apparatus that may possibly be utilized or possibly adapted for use in at least one possible embodiment of the present application may possibly be found in the following U.S. Pat. No. 5,910,739 issued to Stanojevic on Jun. 8, 1999; U.S. Pat. No. 5,999,087 issued to Gunton on Dec. 7, 1999; U.S. Pat. No. 6,016,531 issued to Rixner et al. on Jan. 18, 2000; U.S. Pat. No. 6,020,697 issued to Stenger et al. on Feb. 1, 2000; U.S. Pat. No. 6,020,775 issued to Chevallier on Feb. 1, 2000; and No. 6,038,197 issued to Phillips on Mar. 14, 2000.

Some examples of control valve apparatus that may possibly be utilized or possibly adapted for use in at least one possible embodiment of the present application may possibly be found in the following U.S. Pat. No. 5,406,975 issued to Nakamichi et al. on Apr. 18, 1995; U.S. Pat. No. 5,503,184 issued to Reinartz et al. on Apr. 2, 1996; U.S. Pat. No. 5,706,849 issued to Uchida et al. on Jan. 13, 1998; U.S. Pat. No. 5,975,115 issued to Schwegler et al. on Nov. 2, 1999; U.S. Pat. No. 6,142,445 issued to Kawaguchi et al. on Nov. 7, 2000; and U.S. Pat. No. 6,145,538 issued to Park on Nov. 14, 2000.

Some examples of electric control valves that may possibly be utilized or possibly adapted for use in at least one possible embodiment of the present application may possibly be found in the following U.S. Pat. No. 4,431,160 issued to Burt et al. on Feb. 14, 1984; and U.S. Pat. No. 4,609,176 issued to Powers on Sep. 2, 1986.

Some examples of nozzle structures that may possibly be utilized or possibly adapted for use in at least one possible embodiment of the present application may possibly be found in the following U.S. Pat. No. 6,042,026 issued to Buehler, II on Mar. 28, 2000; U.S. Pat. No. 6,394,366 issued to Adams on May 28, 2002; U.S. Pat. No. 6,402,062 issued to Bendig et al. on Jun. 11, 2002; U.S. Pat. No. 6,616,072 issued to Harata et al. on Sep. 9, 2003; U.S. Pat. No. 6,666,386 issued to Huang on Dec. 23, 2003; and U.S. Pat. No. 6,681,498 issued to Steffan on Jan. 27, 2004.

Some examples of heater arrangements that may possibly be utilized or possibly adapted for use in at least one possible embodiment of the present application may possibly be found in the following U.S. Pat. No. 6,404,421 issued to Meijler et al. on Jun. 11, 2002; U.S. Pat. No. 6,515,264 issued to Toya et al. on Feb. 4, 2003; U.S. Pat. No. 6,548,786 issued to Takizawa et al. on Apr. 15, 2003; U.S. Pat. No. 6,555,796 issued to Cusack on Apr. 29, 2003; U.S. Pat. No. 6,633,727 issued to Henrie et al. on Oct. 14, 2003; and U.S. Pat. No. 6,677,557 issued to Ito et al. on Jan. 13, 2004.

Some examples of temperature sensors or sensor systems that may be used or adapted for use in at least one possible embodiment of the present invention may be found in the following U.S. Pat. No. 5,960,857, issued to inventors Oswalt et al. on Oct. 5, 1999; U.S. Pat. No. 5,942,980, issued to inventors Hoben et al. on Aug. 24, 1999; U.S. Pat. No. 5,881,952, issued to inventor MacIntyre on Mar. 16, 1999; U.S. Pat. No. 5,862,669, issued to inventors Davis et al. on Jan. 26, 1999; U.S. Pat. No. 5,459,890, issued to inventor Jarocki on Oct. 24, 1995; U.S. Pat. No. 5,367,602, issued to inventor Stewart on Nov. 22, 1994; U.S. Pat. No. 5,319,973, issued to inventors Crayton et al. on Jun. 14, 1994; U.S. Pat. No. 5,226,320, issued to inventors Dages et al. on Jul. 13, 1993; U.S. Pat. No. 5,078,123, issued to inventors Nagashima et al. on Jan. 7, 1992; and U.S. Pat. No. 5,068,030, issued to inventor Chen on Nov. 26, 1991.

Some examples of metering valves, which may possibly be utilized or adapted for use in at least one possible embodiment of the present application, may possibly be found in the following U.S. Pat. No. 7,651,014, having the title “Metering valve and device for dispensing a preferably cosmetic liquid,” published on Jan. 26, 2010; U.S. Pat. No. 7,594,516, having the title “Freeze-resistant metering valve,” published on Sep. 29, 2009; U.S. Pat. No. 7,503,344, having the title “High pressure metering valve,” published on Mar. 17, 2009; U.S. Pat. No. 7,487,789, having the title “Gaseous fluid metering valve,” Feb. 10, 2009; U.S. Pat. No. 7,357,278, having the title “Metering valve for fluid substances,” published on Apr. 15, 2008; and U.S. Pat. No. 7,278,625, having the title “Metering valve,” published on Oct. 9, 2007.

Some examples of flow meters and magnetic flow meters, which may possibly be utilized or adapted for use in at least one possible embodiment of the present application, may possibly be found in the following U.S. Pat. No. 7,658,117, having the title “Flow meter using an expanded tube section and sensitive differential pressure measurement,” published on Feb. 9, 2010; U.S. Pat. No. 7,658,114, having the title “Ultrasonic flow meter,” published on Feb. 9, 2010; U.S. Pat. No. 7,637,152, having the title “Flow meter and flow-regulating system using the same,” published on Dec. 29, 2009; U.S. Pat. No. 7,621,188, having the title “Flow meter,” published on Nov. 24, 2009; U.S. Pat. No. 7,587,947, having the title “Magnetic flow meter with selective electrode positioning,” published on Sep. 15, 2009; U.S. Pat. No. 7,581,453, having the title “Ultrasonic flow meter system,” published on Sep. 1, 2009; U.S. Pat. No. 7,574,924, having the title “Magnetic flow meter,” published on Aug. 18, 2009; and U.S. Pat. No. 7,571,655, having the title “Magnetic flow meter with buffering electrodes,” published on Aug. 11, 2009.

Some examples of membrane pumps, which may possibly be utilized or adapted for use in at least one possible embodiment of the present application, may possibly be found in the following U.S. Pat. No. 6,948,918, having the title “Membrane pump with stretchable pump membrane,” published on Sep. 27, 2005; U.S. Pat. No. 6,796,215, having the title “Membrane pump,” published on Sep. 28, 2004; U.S. Pat. No. 6,776,591, having the title “Membrane pump comprising an inlet opening that is controlled by the membrane,” published on Aug. 17, 2004; U.S. Pat. No. 5,533,886, having the title “Membrane pump and method of operating the same,” published on Jul. 9, 1996; U.S. Pat. No. 5,261,798, having the title “Double membrane pump,” published on Nov. 16, 1993; U.S. Pat. No. 4,332,534, having the title “Membrane pump with tiltable rolling piston pressing the membrane,” published on Jun. 1, 1982; and U.S. Pat. No. 3,955,901, having the title “Membrane pump,” published on May 11, 1976.

U.S. patent application Ser. No. 12/707,372, filed on Feb. 17, 2010, having inventors Thomas HEROLD and Roland TOPF, Attorney Docket No. NHL-HOL-273, and title “A BEVERAGE BOTTLE OR CONTAINER FILLING PLANT HAVING A BEVERAGE BOTTLE OR CONTAINER TREATMENT ARRANGEMENT AND A METHOD OF OPERATING A BEVERAGE BOTTLE OR CONTAINER FILLING PLANT,” and its corresponding Federal Republic of Germany Patent Application No. DE 10 2007 039 010.8, filed on Aug. 17, 2007, and International Patent Application No. PCT/EP2008/005828, filed on Jul. 17, 2008, having WIPO Publication No. WO 2009/024218 and inventors Thomas HEROLD and Roland TOPF are hereby incorporated by reference as if set forth in their entirety herein.

U.S. Patent Publication No. 2006/0011262, having the title “Beverage bottling plant for filling bottles with a liquid beverage material having a treatment device for the treatment of bottles,” published on Jan. 19, 2006, is hereby incorporated by reference as if set forth in their entirety herein.

All of the patents, patent applications or patent publications, which were cited in the International Search Report dated Sep. 30, 2008, and/or cited elsewhere are hereby incorporated by reference as if set forth in their entirety herein as follows: DE 40 36 950, having the following English translation of the German title “Supplying sterilising liq. to packing containers—involves through-flow measuring instrument and degasification device to remove bubbles,” published on May 21, 1992; DE 10 2005 030822, having the following German title “Verfahren and Vorrichtung zum Überwachen eines Verdampfers,” published on Jan. 11, 2007; DE 10 2004 059346, having the following English translation of the German title “Object surfaces sterilizing method, involves conducting steam mixture containing water vapor and hydrogen peroxide steam in evacuated sterilization chamber such that mixture forms condensate film immediately on object surfaces,” published on Jun. 1, 2006; DE 199 875 56 186, having the following English translation of the German title “Packaging container sterilization process, comprises using a mixing nozzle to produce a disinfectant—steam mixt which is then sprayed onto the container surfaces,” published on May 23, 2001.

All of the patents, patent applications or patent publications, which were cited in the German Office Action dated Jun. 10, 2008, and/or cited elsewhere are hereby incorporated by reference as if set forth in their entirety herein as follows: WO 2006/128884, having the title “APPARATUS AND METHODS FOR TREATING COMPONENTS OF PACKAGING UNITS, PARTICULARLY BOTTLES AND/OR CAPS,” published on Dec. 7, 2006; DE 103 46 843, having the following German title “Vorrichtung zum Vergasen eines Dekontaminationsmittels,” published on Jun. 24, 2004; and DE 10 2004 030 957, having the following English inflation of the German title “Method of sterilising bottles or similar containers, and steriliser for carrying out the method,” published on Jan. 12, 2006; and DE 10 2005 030822, having the following German title “Verfahren and Vorrichtung zum Überwachen eines Verdampfers,” published on Jan. 11, 2007.

The patents, patent applications, and patent publication listed above in the preceding paragraphs are herein incorporated by reference as if set forth in their entirety. The purpose of incorporating U.S. patents, Foreign patents, publications, etc. is solely to provide additional information relating to technical features of one or more embodiments, which information may not be completely disclosed in the wording in the pages of this application. Words relating to the opinions and judgments of the author and not directly relating to the technical details of the description of the embodiments therein are not incorporated by reference. The words all, always, absolutely, consistently, preferably, guarantee, particularly, constantly, ensure, necessarily, immediately, endlessly, avoid, exactly, continually, expediently, ideal, need, must, only, perpetual, precise, perfect, require, requisite, simultaneous, total, unavoidable, and unnecessary, or words substantially equivalent to the above-mentioned words in this sentence, when not used to describe technical features of one or more embodiments, are not considered to be incorporated by reference herein.

The corresponding foreign and international patent publication applications, namely, Federal Republic of Germany Patent Application No. 10 2007 039 008.6, filed on Aug. 17, 2007, having inventors Daryoush SANGI and Thomas HEROLD, and DE-OS 10 2007 039 91 008.6 and DE-PS 10 2007 039 008.6, and International Application No. PCT/EP2008/006116, filed on Jul. 25, 2008, having WIPO Publication No. WO2009/024247 and inventors Daryoush SANGI and Thomas HEROLD, are hereby incorporated by reference as if set forth in their entirety herein for the purpose of correcting and explaining any possible misinterpretations of the English translation thereof. In addition, the published equivalents of the above corresponding foreign and international patent publication applications, and other equivalents or corresponding applications, if any, in corresponding cases in the Federal Republic of Germany and elsewhere, and the references and documents cited in any of the documents cited herein, such as the patents, patent applications and publications, are hereby incorporated by reference as if set forth in their entirety herein.

The purpose of incorporating the corresponding foreign equivalent patent application(s), that is, PCT/EP2008/006116 and German Patent Application 10 2007 039 008.6, is solely for the purpose of providing a basis of correction of any wording in the pages of the present application, which may have been mistranslated or misinterpreted by the translator. Words relating to opinions and judgments of the author and not directly relating to the technical details of the description of the embodiments therein are not to be incorporated by reference. The words all, always, absolutely, consistently, preferably, guarantee, particularly, constantly, ensure, necessarily, immediately, endlessly, avoid, exactly, continually, expediently, ideal, need, must, only, perpetual, precise, perfect, require, requisite, simultaneous, total, unavoidable, and unnecessary, or words substantially equivalent to the above-mentioned word in this sentence, when not used to describe technical features of one or more embodiments, are not generally considered to be incorporated by reference herein.

Statements made in the original foreign patent applications PCT/EP2008/006116 and DE 10 2007 039 008.6 from which this patent application claims priority which do not have to do with the correction of the translation in this patent application are not to be included in this patent application in the incorporation by reference.

Any statements about admissions of prior art in the original foreign patent applications PCT/EP2008/006116 and DE 10 2007 039 008.6 are not to be included in this patent application in the incorporation by reference, since the laws relating to prior art in non-U.S. Patent Offices and courts may be substantially different from the Patent Laws of the United States.

All of the references and documents, cited in any of the documents cited herein, are hereby incorporated by reference as if set forth in their entirety herein. All of the documents cited herein, referred to in the immediately preceding sentence, include all of the patents, patent applications and publications cited anywhere in the present application.

The description of the embodiment or embodiments is believed, at the time of the filing of this patent application, to adequately describe the embodiment or embodiments of this patent application. However, portions of the description of the embodiment or embodiments may not be completely applicable to the claims as originally filed in this patent application, as amended during prosecution of this patent application, and as ultimately allowed in any patent issuing from this patent application. Therefore, any statements made relating to the embodiment or embodiments are not intended to limit the claims in any manner and should not be interpreted as limiting the claims in any manner.

The details in the patents, patent applications and publications may be considered to be incorporable, at applicant's option, into the claims during prosecution as further limitations in the claims to patentably distinguish any amended claims from any applied prior art.

The purpose of the title of this patent application is generally to enable the Patent and Trademark Office and the public to determine quickly, from a cursory inspection, the nature of this patent application. The title is believed, at the time of the filing of this patent application, to adequately reflect the general nature of this patent application. However, the title may not be completely applicable to the technical field, the object or objects, the summary, the description of the embodiment or embodiments, and the claims as originally filed in this patent application, as amended during prosecution of this patent application, and as ultimately allowed in any patent issuing from this patent application. Therefore, the title is not intended to limit the claims in any manner and should not be interpreted as limiting the claims in any manner.

The abstract of the disclosure is submitted herewith as required by 37 C.F.R. §1.72(b). As stated in 37 C.F.R. §1.72(b):

A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims.

Therefore, any statements made relating to the abstract are not intended to limit the claims in any manner and should not be interpreted as limiting the claims in any manner.

The embodiments of the invention described herein above in the context of the preferred embodiments are not to be taken as limiting the embodiments of the invention to all of the provided details thereof, since modifications and variations thereof may be made without departing from the spirit and scope of the embodiments of the invention.

### AT LEAST PARTIAL NOMENCLATURE

1 Device
2 Bottle
3 Rotor
4 Intake
5 Outlet
6 Treatment head
6.1 Mixing chamber
6.2 Evaporator
6.1.1 Port for sterile air with mixing head nozzle
6.1.2 Port for H₂O₂ with mixing head nozzle
7 Bottle or container carrier
8 Tube
9 Metering and supplying system
10 Reservoir
11 Supply line
12 Control valve
13 Supply line
14 Control valve
15 Connecting line
16 Flow meter
17 Metering valve
18 Metering and dosing unit
19 Control unit
20 Distributing line
21 Gas separator
N₁₀ Level

1-40. (canceled)

41. A method of operating a beverage bottling plant for filling beverage bottles with liquid beverage material, said method comprising the steps of:
conveying beverage bottles to be filled to a first star wheel structure of a rotary beverage bottle filling machine;
moving beverage bottles into said beverage bottle filling machine;
moving beverage bottles with a rotor of said beverage bottle filling machine and substantially simultaneously filling beverage bottles with liquid beverage material;
said step of filling beverage bottles with liquid beverage material comprising:
supplying liquid beverage material to said beverage bottle filling machine from a supply of liquid beverage material; and
dispensing liquid beverage material from a plurality of beverage bottle filling elements into beverage bottles to be filled;
moving filled beverage bottles with a second star wheel structure of said beverage bottle filling machine out of said beverage bottle filling machine and into a second conveyor arrangement;
conveying filled beverage bottles from said beverage bottle filling machine to a first star wheel structure of a beverage bottle closing machine;
moving beverage bottles into said beverage bottle closing machine;
moving beverage bottles with a rotor of said beverage bottle closing machine and substantially simultaneously closing filled beverage bottles;
said step of closing filled beverage bottles comprising placing closures on filled beverage bottles with a plurality of closing devices;
moving closed beverage bottles with a second star wheel structure of said beverage bottle closing machine out of said beverage bottle filling machine;
prior to filling beverage bottles, conveying beverage bottles to a first star wheel structure of a rotary beverage bottle treatment machine;
moving beverage bottles into said rotary beverage bottle treatment machine;
moving beverage bottles with a rotor of said rotary beverage bottle treatment machine and substantially simultaneously treating beverage bottles with a treatment medium;
said rotary beverage bottle treatment machine comprising:
a storage tank being configured and disposed to store treatment medium;
a plurality of treatment heads being disposed about the periphery of said rotor of said rotary beverage bottle treatment machine and being configured to introduce sterile air and treatment medium into beverage bottles;
a connecting line arrangement being configured and disposed to connect said storage tank to each of said plurality of treatment heads; and
said connecting line arrangement comprising a plurality of connecting lines;
each connecting line being configured and disposed to connect one treatment head of said plurality of treatment heads to said storage tank;
said method further comprising connecting one treatment head of said plurality of treatment heads to said storage tank with each connecting line;
each connecting line being sufficiently short to minimize decomposition of the treatment medium in each connecting line during operation of said rotary beverage bottle treatment machine;
said method further comprising minimizing decomposition of the treatment medium in each connecting line during operation of said rotary beverage bottle treatment machine;
each connecting line comprising a cross section being sufficiently small to minimize decomposition of the treatment medium in each connecting line during operation of said rotary beverage bottle treatment machine;
said method further comprising conducting treatment medium through each connecting line comprising said sufficiently small cross section and minimizing decomposition of the treatment medium in each connecting line during operation of said rotary beverage bottle treatment machine;
each of said plurality of treatment heads comprising:
a mixing chamber being configured and disposed to mix sterile air and treatment medium;
a tube being configured and disposed to introduce sterile air and treatment medium into beverage bottles;
a sterile air port being connected to said mixing chamber and being configured to provide sterile air to said mixing chamber;
a treatment medium port being connected to said mixing chamber and being configured to provide treatment medium to said mixing chamber; and
an evaporator being connected to said mixing chamber and being configured to receive mixed sterile air and treatment medium from said mixing chamber;
said method further comprising the steps of:
providing sterile air, through said sterile air port, in said mixing chamber;
providing treatment medium, through said treatment medium port, in said mixing chamber;
mixing sterile air and treatment medium in said mixing chamber;
receiving mixed sterile air and treatment medium, from said mixing chamber, in said evaporator;
evaporating mixed sterile air and treatment medium in said evaporator; and
introducing evaporated, mixed sterile air and treatment medium into beverage bottles with said tube;
each connecting line of said plurality of connecting lines comprising an electrically controlled valve, which electrically controlled valve is configured and disposed to alternate between an open position and a closed position;
said method further comprising alternating said electrically controlled valve between an open position and a closed position;
each said electrically controlled valve being directly connected to its corresponding one of said plurality of treatment heads, to provide a minimal distance between each electrically controlled valve and a mixing chamber of its corresponding one of said plurality of treatment heads;
said step of treating beverage bottles comprising:
pressurizing said storage tank to produce a flow of treatment medium into said connecting line arrangement and to produce a pressure at said plurality of treatment heads of said rotary beverage bottle treatment machine;
flowing treatment medium through said connecting line arrangement;
flowing treatment medium into said plurality of treatment heads at a flow rate which is sufficiently high to minimize decomposition of the treatment medium in each connecting line and each treatment head during operation of said rotary beverage bottle treatment machine and maximize accuracy of the amount of treatment medium flowed into said treatment heads.

42. The method according to claim 41, wherein:
each connecting line of said plurality of connecting lines comprises a flow meter being configured and disposed to measure flow of treatment medium through each connecting line;
said method further comprises measuring the flow of treatment medium, with said flow meter, through each connecting line of said plurality of connecting lines;
said flow meter comprises a magnetic induction flow meter;
said method further comprises measuring the flow of treatment medium, with said magnetic induction flow meter, through each connecting line of said plurality of connecting lines;
said storage tank comprises a container configured to be pressurized with overpressure or metering pressure;
said method further comprising pressurizing said storage tank with overpressure or metering pressure;
each connecting line of said connecting line arrangement comprises a length, which length is approximately one and one half meters to approximately two meters;
said method further comprises minimizing decomposition of treatment medium in each connecting line comprising said length of approximately one and one half meters to approximately two meters during operation of said treatment arrangement;
each connecting line of said plurality of connecting lines, disposed between said storage tank and each of said plurality of treatment heads, comprises a cross section of approximately one and a half millimeters over at least a portion of said length;
said method further comprises minimizing decomposition of treatment medium in each connecting line of said plurality of connecting lines comprising said cross section of approximately one and a half millimeters of at least a portion of said length, disposed between said storage tank and each of said plurality of treatment heads, during operation of said treatment arrangement;
one of (A) and (B):
(A) each connecting line of said plurality of connecting lines, disposed between each of said plurality of treatment heads and said storage tank is configured to house treatment medium for a maximum of sixty seconds; and
said method further comprises conducting treatment medium through said plurality of connecting lines for a maximum of sixty seconds;
(B) each connecting line of said plurality of connecting lines, disposed between each of said plurality of treatment heads and said storage tank is configured to house treatment medium for a maximum of forty seconds; and
said method further comprises conducting treatment medium through said plurality of connecting lines for a maximum of forty seconds;
one of (C), (D), and (E):
(C) each said electrically controlled valve is configured to have a switching time of less than ninety milliseconds; and
said method further comprises operating each said electrically controlled valve with a switching time of less than ninety milliseconds;
(D) each said electrically controlled valve is configured to have a switching time of less than fifty milliseconds; and
said method further comprises operating each said electrically controlled valve with a switching time of less than fifty milliseconds;
(E) each said electrically controlled valve is configured to have a switching time of less than one millisecond; and
said method further comprises operating each said electrically controlled valve with a switching time of less than one millisecond.

43. A method of operating a treatment arrangement for treating packaging items, such as beverage bottles, containers, cans, kegs, tubes, and package materials, in combination with a packaging item filling plant, said method comprising the steps of:
conveying packaging items to said treatment arrangement;
moving packaging items into said treatment arrangement;
treating packaging items with a treatment medium;
said step of treating packaging items comprising:
storing treatment medium in a storage arrangement being configured and disposed to store treatment medium therein;
pressurizing treatment medium to produce a flow of treatment medium into a connecting line arrangement and to produce a pressure at a plurality of treatment heads of said treatment arrangement;
flowing treatment medium through said connecting line arrangement;
discharging treatment medium with a discharge arrangement, which discharge arrangement is configured and disposed to introduce air and treatment medium into containers;
said connecting line arrangement comprises a plurality of connecting lines;
each connecting line of said plurality of connecting lines corresponding to one of said plurality of treatment heads;
each connecting line of said plurality of connecting lines comprising an electrically controlled valve, which electrically controlled valve is configured and disposed to alternate between an open position and a closed position;
said method further comprising alternating said electrically controlled valve between an open position and a closed position; and
said method further comprising operating each electrically controlled valve, which electrically controlled valve is directly connected to its corresponding one of said plurality of treatment heads, to provide a minimal distance between each electrically controlled valve and its corresponding one of said plurality of treatment heads, to minimize decomposition of the treatment medium in each connecting line between each electrically controlled valve and its corresponding one of said plurality of treatment heads during operation of said treatment arrangement, and maximize accuracy of the amount of treatment medium flowed into said treatment heads;
said method further comprising operating said container filling plant;
said step of operating said container filling plant comprising:
filling containers with a container filling machine being configured and disposed to fill containers in said container filling plant; and
closing containers with a container closing machine being configured and disposed to close containers in said container filling plant.

44. The method according to claim 43, wherein said method further comprises flowing treatment medium into said plurality of treatment heads over a distance which is sufficiently short and at a flow rate which is sufficiently high to minimize decomposition of the treatment medium in each connecting line during operation of said treatment arrangement and maximize accuracy of the amount of treatment medium flowed into said treatment heads.

45. The method according to claim 44, wherein each said connecting line comprises a cross section being sufficiently small to minimize decomposition of treatment medium in each connecting line during operation of said treatment arrangement.

46. The method according to claim 45, wherein said method further comprises conducting treatment medium through each said connecting line comprising said sufficiently small cross section and minimizing decomposition of the treatment medium in each connecting line during operation of said treatment arrangement.

47. The method according to claim 46, wherein:
each of said plurality of treatment heads comprises:
a mixing chamber being configured and disposed to mix air and treatment medium;
said discharge arrangement, which discharge arrangement comprises a tube being configured and disposed to introduce air and treatment medium into packaging items;
an air port being connected to said mixing chamber and being configured to provide air to said mixing chamber;
a treatment medium port being connected to said mixing chamber and being configured to provide treatment medium to said mixing chamber; and
an evaporator being connected to said mixing chamber and being configured to receive mixed air and treatment medium from said mixing chamber;
said method further comprising the steps of:
providing sterile air, with said sterile air port, in said mixing chamber;
providing treatment medium, with said treatment medium port, in said mixing chamber;
mixing sterile air and treatment medium in said mixing chamber;
receiving mixed sterile air and treatment medium, from said mixing chamber, in said evaporator;
evaporating mixed sterile air and treatment medium in said evaporator; and
flowing evaporated, mixed sterile air and treatment medium to packaging items with said tube;
said treatment arrangement comprises a flow meter configured and disposed to measure the flow of treatment medium through each connecting line; and
said method further comprises measuring the flow of treatment medium, with said flow meter, through each connecting line of said plurality of connecting lines.

48. The method according to claim 47, wherein:
said flow meter comprises a magnetic induction flow meter;
said method further comprises measuring the flow of treatment medium, with said magnetic induction flow meter, through each connecting line of said plurality of connecting lines;
said storage arrangement comprises a container configured to be pressurized with overpressure or metering pressure; and
said method further comprises pressurizing said storage arrangement with overpressure or metering pressure.

49. The method according to claim 48, wherein:
each connecting line of said connecting line arrangement comprises a length, which length is approximately one and one half meters to approximately two meters; and
said method further comprises minimizing decomposition of treatment medium in each connecting line comprising said length of approximately one and one half meters to approximately two meters during operation of said treatment arrangement.

50. The method according to claim 49, wherein:
each connecting line of said plurality of connecting lines, disposed between said storage arrangement and each of said plurality of treatment heads, comprises a cross section of approximately one and a half millimeters over at least a portion of said length; and
said method further comprises minimizing decomposition of treatment medium in each connecting line of said plurality of connecting lines, disposed between said storage arrangement and each of said plurality of treatment heads comprising said cross section of approximately one and a half millimeters of at least a portion of said length, during operation of said treatment arrangement.

51. The method according to claim 50, wherein:
one of (A) and (B):
(A) each connecting line of said plurality of connecting lines, disposed between each of said plurality of treatment heads and said storage arrangement is configured to house treatment medium for a maximum of sixty seconds; and
said method further comprises conducting treatment medium through said plurality of connecting lines for a maximum of sixty seconds;
(B) each connecting line of said plurality of connecting lines, disposed between each of said plurality of treatment heads and said storage arrangement is configured to house treatment medium for a maximum of forty seconds; and
said method further comprises conducting treatment medium through said plurality of connecting lines for a maximum of forty seconds;
one of (C), (D), and (E):
(C) each said electrically controlled valve is configured to have a switching time of less than ninety milliseconds; and
said method further comprises operating each said electrically controlled valve with a switching time of less than ninety milliseconds;
(D) each said electrically controlled valve is configured to have a switching time of less than fifty milliseconds; and
said method further comprises operating each said electrically controlled valve with a switching time of less than fifty milliseconds;
(E) each said electrically controlled valve is configured to have a switching time of less than one millisecond; and
said method further comprises operating each said electrically controlled valve with a switching time of less than one millisecond.

52. A treatment arrangement being configured to treat packaging items, such as beverage bottles, containers, cans, kegs, tubes, and package materials, in combination with a packaging item filling plant, said treatment arrangement comprising:
a conveying arrangement being configured to convey packaging items to said treatment arrangement and move packaging items into said treatment arrangement;
a packaging item treating arrangement being configured treat to packaging items with a treatment medium;
a storage arrangement being configured and disposed to store treatment medium therein;
a connecting line arrangement comprising a plurality of connecting lines;
a plurality of treatment heads being configured and disposed to introduce treatment medium to packaging items;
each connecting line of said plurality of connecting lines being connected to a corresponding one of said plurality of treatment heads;
each connecting line comprising a discharging arrangement being configured to discharge air and treatment medium into packaging items;
each connecting line of said plurality of connecting lines comprising an electrically controlled valve, which electrically controlled valve is configured and disposed to alternate between an open position and a closed position;
each said electrically controlled valve being directly connected to its corresponding one of said plurality of treatment heads, to provide a minimal distance between each electrically controlled valve and its corresponding one of said plurality of treatment heads, to minimize decomposition of the treatment medium in each connecting line between each electrically controlled valve and its corresponding one of said plurality of treatment heads during operation of said treatment arrangement, and maximize accuracy of the amount of treatment medium flowed into said treatment heads;
said treatment arrangement being disposed in said packaging item filling plant, which packaging item filling plant comprises:
a packaging item filling machine being configured and disposed to fill packaging items in said packaging item filling plant; and
a packaging item closing machine being configured and disposed to close packaging items in said packaging item filling plant.

53. The treatment arrangement according to claim 52, wherein said pressurizing arrangement is configured and disposed to pressurize treatment medium to produce a flow rate which is sufficiently high to minimize decomposition of the treatment medium in each connecting line during operation of said treatment arrangement and maximize accuracy of the amount of treatment medium flowed into said treatment heads.

54. The treatment arrangement according to claim 53, wherein each said connecting line comprises a cross section being sufficiently small to minimize decomposition of treatment medium in each connecting line during operation of said treatment arrangement.

55. The treatment arrangement according to claim 54, wherein each said connecting line is configured to be sufficiently short to minimize decomposition of the treatment medium in each connecting line during operation of said treatment arrangement and maximize accuracy of the amount of treatment medium flowed into said treatment heads.

56. The treatment arrangement according to claim 55, wherein:
each of said plurality of treatment heads comprises:
a mixing chamber being configured and disposed to mix air and treatment medium;
said discharge arrangement, which discharge arrangement comprises a tube being configured and disposed to introduce air and treatment medium into packaging items;
an air port being connected to said mixing chamber and being configured to provide air to said mixing chamber;
a treatment medium port being connected to said mixing chamber and being configured to provide treatment medium to said mixing chamber; and
an evaporator being connected to said mixing chamber and being configured to receive mixed air and treatment medium from said mixing chamber; and
said treatment arrangement comprises a flow meter configured and disposed to measure the flow of treatment medium through each connecting line.

57. The treatment arrangement according to claim 56, wherein:
said flow meter comprises a magnetic induction flow meter;
said storage arrangement comprises a container configured to be pressurized with overpressure or metering pressure.

58. The treatment arrangement according to claim 57, wherein each connecting line of said connecting line arrangement comprises a length, which length is approximately one and one half meters to approximately two meters.

59. The treatment arrangement according to claim 58, wherein each connecting line of said plurality of connecting lines, disposed between said storage arrangement and each of said plurality of treatment heads, comprises a cross section of approximately one and a half millimeters over at least a portion of said length.

60. The treatment arrangement according to claim 59, wherein:
one of (A) and (B):
(A) each connecting line of said plurality of connecting lines, disposed between each of said plurality of treatment heads and said storage arrangement is configured to house treatment medium for a maximum of sixty seconds; and
(B) each connecting line of said plurality of connecting lines, disposed between each of said plurality of treatment heads and said storage arrangement is configured to house treatment medium for a maximum of forty seconds; and
one of (C), (D), and (E):
(C) each said electrically controlled valve is configured to have a switching time of less than ninety milliseconds; and
(D) each said electrically controlled valve is configured to have a switching time of less than fifty milliseconds; and
(E) each said electrically controlled valve is configured to have a switching time of less than one millisecond.
